(19) United States
(12) Patent Application Publication
(10) Pub. No.: US 2003/0114906 A1
(43) Pub. Date: Jun. 19, 2003

(54) Apparatus and methods for deploying cardiac electrodes

(75) Inventors: Robert S. Booker III, San Clemente, CA (US); Reynaldo B. Halili JR., Carlsbad, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TheraCardia, Inc., San Clemente, CA

(21) Appl. No.: 10/023,567

(22) Filed: Dec. 17, 2001

(51) Int. Cl. A61N 1/05

(52) U.S. Cl. 607/122

(57) ABSTRACT

A cardiac electrode deployment device comprises a support and an electrode structure deployable from the support. The electrode structure includes a planar region and a conformable, raised center region, wherein the electrode surfaces on the planar region and on the center region are electrically isolated from each other. The electrode structure is configured to engage against an outer surface of the heart, such as the pericardium, in order to provide electrical contact with the heart for EKG monitoring of the heart, delivery of pacing energy, and/or other low power applications. Usually, the electrode structure will also be suitable for performing direct cardiac massage.

### BACKGROUND OF THE INVENTION

1. Field of the Invention

The present invention relates generally to medical devices and methods. More particularly, the present invention relates to devices and methods for performing minimally invasive direct cardiac monitoring, pacing, and massage.

Sudden cardiac arrest is a leading cause of death in most industrial societies. While in many cases it is possible to re-establish cardiac function, irreversible damage to vital organs, particularly the brain and the heart itself, will usually occur prior to restoration of normal cardiac activity.

A number of techniques have been developed to provide artificial circulation of blood to oxygenate the heart and brain during the period between cardiac arrest and restoration of normal cardiac activity. Prior to the 1960's, open chest cardiac massage (OCM) was a standard treatment for sudden cardiac arrest. Open chest cardiac massage, as its name implies, involved opening a patient's chest and manually squeezing the heart to pump blood to the body. In the 1960's, closed chest cardiac massage (CCM) where the heart is externally compressed through the chest wall became the standard of treatment. When CCM is combined with airway support, it is known as cardiopulmonary resuscitation (CPR). CPR has the advantage that it is much less invasive than OCM and can be performed by less skilled individuals. It has the disadvantage, however, that it is not generally effective at pumping blood for more than a few minutes. In particular, the medical literature shows that CCM provides significantly less cardiac output, neuroperfusion, and cardiac perfusion than achieved with OCM.

Of particular interest to the present invention is the recent introduction of devices for performing minimally invasive direct cardiac massage. Such devices and methods are described in co-pending application Ser. Nos. 09/087,665 filed May 29, 1998, now U.S. Pat. No. 6,200,280; 60/111,934 filed Dec. 11, 1998 (now abandoned); 09/344,440 filed Jun. 25, 1999; 09/356,064 filed Jul. 19, 1999; 09/801,421 filed Mar. 7, 2001; and 09/898,701 filed Jul. 2, 2001, assigned to the assignee of the present application. The full disclosures of each of these prior patents and/or applications are incorporated herein by reference. Specifically, such methods rely on introducing a plurality of struts, an expansible flared bell structure, a laterally oriented expansible structure, or other expandable member to engage the heart through a small incision through an intercostal space to a region over the pericardium or other heart surface. The heart may then be pumped by directly engaging the deployed expansible structure against the pericardium to repeatably compress the heart, typically by reciprocating a shaft attached to the member. Additional minimally invasive direct cardiac massage devices and methods are described in U.S. Pat. Nos. 5,582,580; 5,571,074; and 5,484,391 issued to Buckman, Jr. et al. and U.S. Pat. Nos. 5,931,850; 5,683,364; and 5,466,221 issued to Zadini et al., licensed to the assignee of the present application. Data shows that such devices are able to achieve significantly improved hemodynamic parameters when compared to conventional closed chest cardiac massage.

Patients in sudden cardiac arrest have various states of dysfunction including ventricular fibrillation, ventricular bradycardia, ventricular tachycardia, pulseless electromechanical activity, electromechanical dissociation, and asystole. Thus, to properly evaluate patients in sudden cardiac arrest, it is necessary to monitor electrical heart function by performing an electrocardiogram (ECG or EKG). EKG monitoring may also assess the need for defibrillation energy to effect electrical cardioversion to a more stable heart rhythm for those patients suffering from a heart arrhythmia. Pacing of the heart may also be desirable to regulate the rhythm of the heartbeat.

Cardiac electrode deployment devices for defibrillating, pacing, monitoring, and massage have been described by Buckman et al. in co-pending application Ser. No. 09/406,050, and Masson et al. in co-pending application Ser. No. 09/502,311, both assigned to the assignee of the present application. These devices generally deploy an electrode structure having multiple flat regions that may be electrically isolated from each other against the heart to apply defibrillation energy to the heart though the electrode structure. While this electrode structure holds great promise, there may be circumstances where alternative electrode structures would be advantageous, particularly for low power applications such as EKG monitoring, pacing, and the like.

For these reasons, it would be desirable to provide improved devices, systems, methods, and kits for monitoring and pacing patients in sudden cardiac arrest. In particular, it would be desirable to provide such improved devices and methods which enable and facilitate monitoring of a patient's heart rhythm and/or cardiac pacing while simultaneously performing direct cardiac massage in such patients. It would be further desirable if the electrode structure(s) used to perform such procedures could provide bipolar electrode surfaces with enhanced signal acquisitions and improved surface contact with the heart. Additionally, it would be desirable if such electrode structure(s) could be provided on a device suitable for also performing direct cardiac massage so as to provide a convenient, self-contained system. The devices and systems should also be simple and less costly to manufacture and produce. At least some of these objectives will be met by the invention described hereinafter.

2. Description of the Background Art

Cardiac electrode deployment devices for defibrillating, pacing, monitoring, and massage have been described in co-pending application Ser. Nos. 09/406,050 and 09/502,311, both assigned to the assignee of the present application. Cardiac monitoring, pacing, and defibrillating devices are also described in U.S. Pat. Nos. 4,596,252; 4,603,705; 5,269,319; 5,388,586; 5,391,200; 5,509,924; 5,800,334; 5,904,711; 5,910,124; 6,253,099; 6,263,238; and 6,280,463. External dome shaped electrodes are described in U.S. Pat. No. 4,589,287. U.S. Pat. Nos. 5,484,391, 5,582,580; and 5,571,074 to Buckman, Jr. et al. and U.S. Pat. Nos. 5,466,221, 5,683,364, 5,931,850, and 5,978,714 to Zadini et al., licensed to the assignee of the present application, describe devices and methods for minimally invasive direct cardiac massage through an intercostal space, which optionally incorporate electrodes for defibrillation, pacing, EKG monitoring, and cardioversion. Additional devices and methods for minimally invasive direct cardiac massage are described in co-pending U.S. patent application Ser. No. 09/087,665 filed May 29, 1998, now U.S. Pat. No. 6,200,280; U.S. Provisional Patent Application No. 60/111,934 filed Dec. 11, 1998 (now abandoned); U.S. patent application Ser. Nos. 09/344,440 filed Jun. 25, 1999; 09/356,064 filed Jul. 19, 1999; 09/801,421 filed Mar. 7, 2001; 09/895,844 filed Jun. 29, 2001; and 09/898,701 filed Jul. 2, 2001, assigned to the assignee of the present application. Published PCT application WO 98/05289 and U.S. Pat. Nos. 5,466,221 and 5,385,528 describe an inflatable and other devices for performing direct cardiac massage. Devices and methods for establishing intercostal access are described in co-pending U.S. patent application Ser. Nos. 09/768,041 filed Jan. 22, 2001; 09/______ (Attorney Docket 018803-001710US); and 09/______ (Attorney Docket 018803-002300US) filed Oct. 23, 2001, assigned to the assignee of the present application. U.S. Pat. No. 3,496,932 describes a sharpened stylet for introducing a cardiac massage device to a space between the sternum and the heart. Cardiac assist devices employing inflatable cuffs and other mechanisms are described in U.S. Pat. Nos. 5,256,132; 5,169,381; 4,731,076; 4,690,134; 4,536,893; 4,192,293; 4,048,990; 3,613,672; 3,455,298; and 2,826,193. Dissectors employing inflatable components are described in U.S. Pat. Nos. 5,730,756; 5,730,748; 5,716,325; 5,707,390; 5,702,417; 5,702,416; 5,694,951; 5,690,668; 5,685,826; 5,667,520; 5,667,479; 5,653,726; 5,624,381; 5,618,287; 5,607,443; 5,601,590; 5,601,589; 5,601,581; 5,593,418; 5,573,517; 5,540,711; 5,514,153; and 5,496,345. Use of a direct cardiac massage device of the type shown in the Buckman, Jr. et al. patents is described in Buckman et al. (1997) Resuscitation 34:247-253, (1995) Resuscitation 29:237-248, and (2001) Resuscitation 50:257-262.

The full disclosures of each of the above references are incorporated herein by reference.

### BRIEF SUMMARY OF THE INVENTION

The present invention provides improved devices, systems, methods, and kits for monitoring and pacing patients suffering from cardiac failure, such as ventricular fibrillation, ventricular bradycardia, ventricular tachycardia, pulseless electromechanical activity, electromechanical dissociation, and asystole. Electrical heart function may be monitored by performing an electrocardiogram (ECG or EKG), which in turn may also assess the need for defibrillation energy to effect electrical cardioversion to a more stable heart rhythm. Pacing of the heart may also be performed to regulate the rhythm of the heartbeat. The present invention is particularly useful for patients in sudden cardiac arrest and even more particularly useful for patients in sudden cardiac arrest who are simultaneously undergoing minimally invasive direct cardiac massage. The present invention provides devices which have improved electrode structures that ensure enhanced signal acquisitions and improved surface contact with the heart. The present invention still further provides devices, systems, and kits which are optimized for performing monitoring, pacing, and/or direct cardiac compression in a convenient self-contained unit. Although preferred for such low power electrical functions, it is possible to use the present devices for defibrillation by modifying operations, as described in more detail below, so that there is need no need to switch devices during a treatment procedure.

In a first aspect of the present invention, a cardiac electrode deployment device comprises a support and an electrode structure deployable from the support. The electrode structure includes a planar region and a conformable, raised center region, wherein the electrode surfaces on the planar region and on the center region are electrically isolated from each other. It will be appreciated that the deployment device is not limited to a single electrode structure, but may include multiple electrode structures having planar and/or raised center regions that are isolated from each other. The electrode structure is configured to engage against an outer surface of the heart, such as the pericardium, in order to provide electrical contact with the heart.

The electrode structure comprises an electrically conductive base and an electrically conductive dome attached to an electrically insulated spacer isolating the dome from the base. The electrically conductive base may be a compliant web, such as a conductive fabric, having an active electrode surface area in the range from 10 cm² to 300 cm², preferably from 32 cm² to 150 cm². The electrically conductive dome may be a soft matrix or mesh attached to and projecting from the web and having an active electrode surface area in the range from 0.06 cm² to 20 cm², preferably from 10 cm² to 20 cm². The insulative spacer, made from rubber, plastic, or like insulative materials, attaches the dome to the compliant web and ensures that the planar and center regions are sufficiently separated and that the dome remains centered. The convex surface of the domed electrode structure is advantageous since its protruding surface can ensure enhanced electrical contact with the heart which in turn ensures reliable EKG and pacing signal acquisitions. The soft matrix of the conductive dome further ensures increased surface contact with the heart, especially in circumstances where the electrode structure is deployed at odd angles against the heart. Preferably, the electrode structure on the deployment device will be collapsible, i.e., it will be capable of being shifted between a low profile configuration suitable for intercostal introduction to a region over the patient's heart and to an open configuration where the entire active support area of the electrode surfaces can be engaged against the heart.

The electrode surfaces of the electrode structure can comprise a plurality of electrically isolated segments which can be separately but simultaneously energized from opposite poles of an external power supply controller, allowing bipolar functioning as described in more detail below. The electrode support will usually include separate electrical conduction paths for connecting the isolated segments of the electrode structure to the external power supply controller. Preferably, the support comprises a first electrically conductive path for connecting the electrode surface on the planar region to the external power supply and a second electrically conductive path isolated from the first path for connecting the electrode surface of the center region to the external power supply controller.

The support may be any assembly, structure, system, or other mechanical framework which is suitable for positioning and manipulating the electrode structure so that it can engage the heart. Most simply, the support could be a simple handle or shaft having a proximal end and a distal end with the electrode structure attached at a distal end thereof. In an exemplary embodiment, the compliant web is mounted on a plurality of struts which are reciprocatably attached to the distal end of the shaft. The struts are retractable to a radially contracted configuration and advancable along arcuate, diverging paths to define a surface which non-traumatically engages the pericardium to compress the heart when advanced against the pericardium. The struts will typically be composed of a resilient material, more typically be composed of a shape memory alloy, such as nickel titanium alloy, and will usually be formed to deploy radially outwardly and advance along the desired arcuate, diverging paths as they are advanced from a constraining member, usually a tubular sheath. The struts may be advanced and retracted relative to the sheath using any suitable mechanical system, typically a shaft which reciprocates together with the struts through a lumen of the sheath. In some instances, it will be desirable to provide at least some of the struts with a temperature-responsive memory so that the shape of the struts will change in response to a transition from room temperature to body temperature and/or in response to an induced temperature change after they have been deployed, e.g., by electrically heating or cooling the struts and/or infusing a heated or cooled medium into the space surrounding the struts.

The compliant web or conductive fabric will be secured to the struts to advance the electrode surfaces when the struts are advanced. In this embodiment, the compliant web is supported solely by the struts and the soft, conductive dome is supported solely by the web. Hence, once the struts or other heart-engaging member is deployed, cardiac monitoring, pacing, and massage can be performed by energizing the electrode structure and by simple manual pumping or reciprocation of the shaft or alternatively by automatic or powered systems for actuating the shaft. Optionally, the device may simply sense and pace the heart without any pumping action.

In some instances, the deployment device may further incorporate a non-conductive, fixed rod which is advancable from a distal end of the shaft to urge the dome forward (i.e. to protrude distally of the compliant web) as the electrode surfaces are deployed against the heart to maximize electrode surface contact with the heart. In such an embodiment, the fixed rod is coupleable to a back region of the dome, the compliant web, and the distal end of the shaft. Additionally or alternatively, the deployment device may further include a spring mechanism which is advancable from the distal end of the shaft to urge the dome forward. For example, the spring may be attached to the distal end of the shaft to provide a spring loaded advancement of the fixed rod which in turn urges the dome to protrude distally of the compliant web.

In another aspect of the present invention, a cardiac electrode deployment device may comprise a support having a proximal end, a distal end, and a blunt tip. A first electrode structure may be deployable from the distal end of the support, wherein the first electrode structure includes a planar region. A second electrode structure may be attached to the blunt tip, wherein the second electrode structure has a conformable, raised center region, and electrode surfaces on the first and second electrode structures are electrically isolated from each other. In this embodiment, the first electrode structure comprises an electrically conducive base, such as a compliant web or fabric as mentioned above. The second electrode structure comprises an electrically conductive dome, such as a soft matrix or mesh, disposed over the blunt tip. Most simply, the second electrode may comprise the blunt tip, wherein the blunt tip is formed from a soft, biocompatible foam core covered with a conductive material or entirely formed from a compressible, conductive material. Hence, the blunt tip advantageously allows for both intercostal dissection and sensing or pacing of the heart.

The first electrode structure may comprise a compliant web secured to a plurality of struts reciprocatably attached to the distal end of the shaft. The struts are retractable to a radially contracted configuration and advancable along arcuate, diverging paths to deploy the first electrode surface to non-traumatically engage the heart when advanced there against and will have characteristics as described above. The compliant web is secured to the struts to advance the first electrode surface when the struts are advanced. The compliant web in this embodiment is supported solely by the struts and the dome is supported solely by the blunt tip, wherein the blunt tip extends from the most distal end of the shaft by a rod. The blunt tip may be formed from a soft, biocompatible foam core or may optionally be entirely formed from soft conductive mesh. The deployment device of the present invention may further incorporate a force gauge, accelerometer, impedance sensor, piezoelectric crystal, or oximeter into the blunt tip or dome to allow monitoring of a variety of other characteristics (e.g. monitor compression force applied by the device).

The present invention still further provides systems comprising a support, an electrode structure deployable from the support, and a power supply controller. The electrode structure, as described above, includes a planar region and a conformable, raised center region, wherein electrode surfaces on the planar region and on the center region are electrically isolated from each other. The support comprises a first electrically conductive path for connecting the electrode surface on the planar region to the external power supply controller and a second electrically conductive path isolated from the first path for connecting the electrode surface of the center region to the external power supply controller. The power supply may additionally include circuitry and/or programming necessary for EKG monitoring, pacing, and the like. Optionally, the systems may further comprise a paired counter electrode for performing defibrillation treatments, as described in more detail hereinbelow.

In a further aspect, methods according to the present invention comprise electrically contacting a patient's heart. An electrode structure is percutaneously introduced against the heart. A first electrically conductive path is established to the heart through a first electrode surface on a planar region of the electrode structure. A second electrically conductive path is established to the heart through a second electrode surface on a raised center region of the electrode structure, wherein the first and second electrode surfaces are electrically isolated from each other. An electrical circuit is then established between the first and second electrically conductive paths. Establishing an electrical circuit may comprise taking an EKG of the heart to assess electrical functioning of the heart and/or pacing of the heart to regulate the rhythm of the heartbeat. Preferably, EKG sensing and pacing will be carried out in a bipolar fashion where the first and second isolated electrode surfaces are positioned on or within the heart or inner thoracic cavity and energy is applied through the isolated electrode surfaces, wherein said surfaces can be separately but simultaneously energized from opposite poles of the power supply controller. EKG sensing and pacing may optionally be carried out in a unipolar fashion where the electrode structure (e.g. planar and/or center regions) contacted against the heart, is attached to one pole of the power supply and a counter or dispersive electrode contacted against a patient's front or back torso, is attached to the other pole to provide the necessary current return path. For unipolar pacing, the dispersive or counter electrode may be positioned anywhere on a patient's body as it acts as a ground and is not position sensitive.

Establishing the first electrically conductive path comprises engaging an electrically active, compliant web against the heart and establishing the second electrically conductive path comprises engaging a soft dome-like matrix coupled to and projecting from the web against the heart. The dome like-matrix may additionally be advanced beyond the web with any of the mechanisms discussed above. Alternatively, the first electrically conductive path may comprise engaging an electrically conductive compliant web against the heart and establishing the second electrically conductive path may comprise engaging a soft dome-like matrix disposed over a blunt tip against the heart. In the latter case, the blunt tip may facilitate introduction of the electrode structure by bluntly dissecting intercostal tissue.

Preferably, the heart will be compressed using an electrode structure contacted and pressed against a surface of the heart, typically the pericardium, where the electrode structure can be used to monitor and pace the heart during compression. Usually, compression is in an anterior-posterior direction, and the electrode structure is preferably percutaneously introduced, as described elsewhere in this application and in the patents and co-pending applications which have been incorporated herein by reference. Most preferably, the electrode structure is introduced intercostally in a low profile configuration and subsequently expanded over the heart in order to deploy the electrode structure in a desired manner. The heart will typically be repetitively compressed in the range 40 to 160 repetitions per minute, preferably from 80 to 120 repetitions per minute.

EKG monitoring may also assess the need for defibrillation energy application to affect electrical cardioversion to a more stable heart rhythm. Usually, defibrillation energy will be applied to the patient in a unipolar fashion, wherein the patient's front or back torso, preferably the back, is engaged with a counter or dispersive electrode and defibrillation energy is applied between the electrode structure (e.g. planar and/or center regions) on the heart and the counter electrode on the back so that direct current countershock can be applied to the heart along an anterior-posterior direction. It will be appreciated that defibrillation generally has a vector effect, and as such the position of the counter electrode does change the performance of the defibrillation electrode system. Hence, the present device may be used for defibrillation by modifying operations. Specifically, a switch on the power supply controller connected to the electrode structure and the counter electrode allows a user to switch the mode of operation between bipolar functioning for sensing or pacing treatment and unipolar functioning for defibrillation treatment. Typically, biphasic defibrillation will require a total amount of energy in the range from 2 joules to about 100 joules, preferably in the range from 20 joules to 50 joules, with a defibrillation threshold of approximately 20 joules. Monophasic defibrillation will usually require a total amount of energy in the range from 2 joules to about 100 joules, preferably in the range from 30 joules to 50 joules, with a defibrillation threshold of approximately 30 joules.

The present invention will also comprise kits including a cardiac electrode deployment device in combination with instructions for use setting forth any of the methods described above.

A further understanding of the nature and advantages of the present invention will become apparent by reference to the remaining portions of the specification and drawings.

### BRIEF DESCRIPTION OF THE DRAWINGS

FIG. 1 is a schematic illustration of a cardiac electrode deployment system constructed in accordance with the principles of the present invention.

FIG. 2 illustrates an exploded view of the electrode structure of FIG. 1 taken along lines 2-2.

FIG. 2A illustrates an alternative electrode structure configuration for the device of FIG. 1.

FIG. 3 illustrates an exemplary electrically conductive fabric comprising conductive and non-conductive threads.

FIGS. 4A-4C illustrate an exemplary cardiac electrode deployment device in its retracted and expanded configurations.

FIGS. 5A and 5B illustrate detailed views of the distal end of the device of FIG. 4C shown with the electrode deployment structure in its open or expanded configuration.

FIGS. 6A-6C illustrate alternative embodiments of the device of FIG. 1.

FIGS. 7A and 7B illustrate another hinged-strut structure in a retracted and deployed configuration, respectively.

FIGS. 8A-8C illustrate use of the device of FIGS. 4A-4C in the simultaneous cardiac compression and electrical monitoring or pacing methods of the present invention.

FIG. 8D illustrates use of the device of FIG. 7A to bluntly dissect intercostal tissue.

FIG. 8E illustrates use of a device having a counter electrode configured to engage a patient's back for defibrillation methods.

FIG. 9 is a flow chart illustrating treatment protocols according to the methods of the present invention.

FIG. 10 illustrates an exemplary kit constructed in accordance with the principles of the present invention.

### DETAILED DESCRIPTION OF THE INVENTION

According to the present invention, devices, systems, methods, and kits are provided for monitoring and pacing patients suffering from cardiac failure. The cardiac failure may be manifested in ventricular fibrillation, ventricular bradycardia, ventricular tachycardia, pulseless electromechanical activity, electromechanical dissociation, and asystole. Electrical heart function may be monitored by performing an electrocardiogram, which in turn may also assess the need for defibrillation or pacing, usually in combination with direct percutaneous cardiac compression. In direct cardiac compression, a cardiac electrode and/or compression structure is contacted against the heart, and such direct contact permits effective monitoring and treatment of the cardiac failure as described in detail below.

The electrode structures may comprise a wide variety of specific designs, but will typically include a planar surface and a raised central surface, wherein electrode surfaces on the planar and center regions are electrically isolated from each other. The electrically conductive surfaces will usually, but not necessarily, be compliant (elastic or non-elastic) so that the surfaces can conform to the heart tissue when the electrode structure is contacted against the heart. The electrically conductive surfaces may be formed entirely from a conductive metal or electrically conductive fibers, e.g., be woven from metal filaments, or may be formed from an electrically insulating backing which is coated with an electrically conductive surface material, typically by plating, sputtering, plasma deposition, or the like. The electrically insulating backing may be formed from a matrix, mesh, fabric, polymeric film, or the like. The electrically conductive surfaces will have areas within the ranges set forth above and provide improved surface contact with the heart which in turn ensures enhanced signal acquisitions. While it is generally preferable that the electrode structures be compliant or otherwise conformable to the heart surface, it is possible in some instances to use electrode structures which are generally rigid and which will cause the heart surface to conform to their geometries when the electrode is pressed against the heart.

The present invention will find its greatest use in minimally invasive procedures where the electrode and/or compression structure is introduced to a region over the heart via a percutaneous access route. A preferred percutaneous access route is intercostal, typically through the fourth or fifth intercostal space and directly over the heart. In such instances, the electrode/compression structure may be introduced in a generally anterior-posterior direction so that direct contact and/or compression of the heart could be achieved by engaging the structure against the heart. More specifically, the electrode/compression structure will usually engage the pericardium covering the heart. For simplicity of explanation, however, the following description will refer to “engaging the heart.” In some cases it might be possible to engage the epicardium directly, but such an approach will be less preferred. Alternatively, in some cases the electrode/compression structure could be introduced via a subxiphoid approach, i.e., from a point below the sternum to a region above the heart.

When the anterior-posterior approach is employed, the handle of the device will preferably be introduced through a left intercostal space in the patient's left rib cage (over the heart), with the handle of the device inclined in the mid-sagittal plane, typically at an angle in the range from 0° to 45°, preferably from 10° to 30°, toward the patient's left side, so that the device compresses the heart toward the patient's spine. The handle may have little or no inclination in the cranial-caudal plane, although some inclination may be required depending on the device entry point in the patient anatomy. If the device is deployed through a right intercostal space, similar angles but reverse orientations would be used.

In most cases, the electrode and/or compression structure will be collapsible, i.e., be shiftable between a low profile configuration where it can easily be introduced either through the intercostal or subxiphoid approach and thereafter deployed at the target region to expand the surface area(s) of the electrode(s) to its desired size. For example, electrodes and compression structures which are formed on or from a film, mesh, matrix, fabric, or other foldable material, may be folded or otherwise collapsed prior to introduction and deployment. In other instances, it would be possible to arrange the electrode/compression structures with discrete joints, hinge regions, or other mechanical features which allow otherwise rigid structures to be folded into a low profile configuration. In still other instances, the electrode/compression structures may be formed as or on an inflatable balloon to effect deployment. Preferably, the electrode/compression structures will be capable of being collapsed to a profile having a width in at least one direction (or diameter when circular) no greater than 50 mm, preferably no greater than 15 mm. When the device is intended for intercostal insertion, it is necessary that it be inserted between adjacent ribs. In that case, an elliptical or oval periphery will have a width along the small axis which is preferably no greater than 20 mm. The size along the long axis is less critical, typically being in the range from 15 mm to 60 mm.

The electrode structures will be utilized and configured to permit EKG/ECG monitoring of the heart. The same transmission lines which connect the isolated region(s) of the electrode structure can be connected to conventional EKG/ECG monitoring circuitry within the power supply controller or other control box. Usually, at least two electrode regions on the electrode structure which contact the heart are used for EKG/ECG monitoring.

The electrode structures may also be utilized for pacing. Pacing requires at least one isolated electrode region on the heart to deliver electrical current pulses to induce heart contraction. Preferably, the pulses are delivered between the planar and raised central electrode surfaces on the heart. The amplitude of such pacing pulses will typically be in the range from 1 mA to 200 mA, usually in the range from 5 mA to 100 mA. The pacing pulse may take the form of any conventional cardiac pacing pulse waveform, e.g., square wave, sine wave, biphasic, monophasic, or other suitable waveform including truncated exponential and combination waveforms. The most common waveform will be the monophasic truncated exponential waveform which is the present standard waveform. In particular embodiments of the present invention, switching or sensing apparatus can be applied to coordinate the delivery of pacing pulses with the heart compression. For example, a motion or other limit switch could be provided to deliver the pacing pulse at a predetermined, repeatable point in the compression cycle which is being induced by direct cardiac massage, usually at the beginning of a compression cycle.

The present invention conveniently provides EKG monitoring, pacing, and massage capabilities through the same electrode structure. The EKG could be used to determine the appropriate timing for pacing the heart. The EKG could also be used to confirm and/or adjust the position of the electrode structure on the heart based on expected waveforms, etc. The EKG could further assess the need for defibrillation energy.

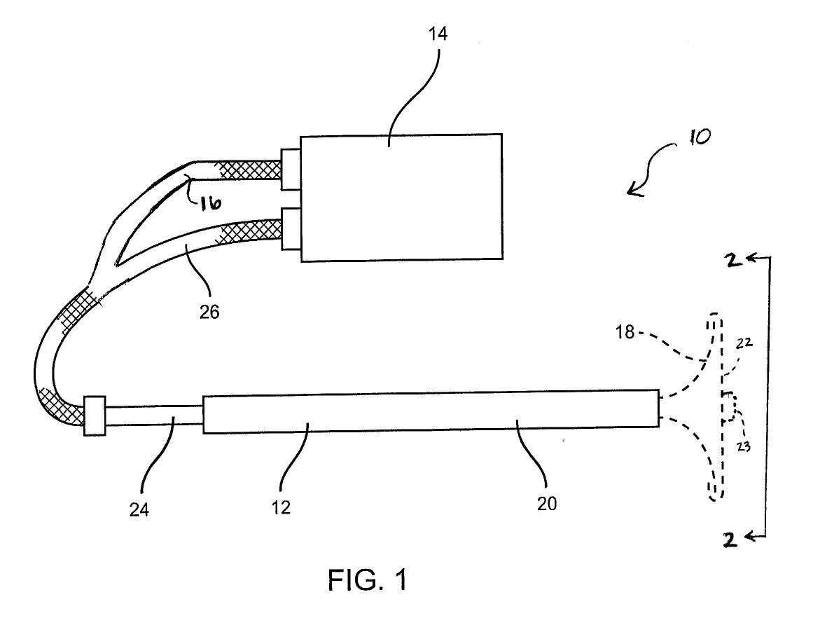

Referring now to FIG. 1, a cardiac electrode deployment device suitable for performing the methods of the present invention will be described. Cardiac electrode deployment device 12 is part of a system 10 which further includes a power supply controller 14. The power supply contains the circuitry necessary for producing EKG monitoring, pacing energy, or other low power applications which can be delivered or sensed by the electrode structure 18 which is shown in its deployed configuration in broken line. The electrode structure 18 generally includes a planar region 22 and a conformable, raised center region 23, wherein electrode surfaces on the planar region 22 and on the center region 23 are electrically isolated from each other. Electrode structure 18 is preferably shiftable between a low profile configuration (where it is drawn rearwardly) into delivery sheath 20 and the deployed configuration shown in broken line. Most simply, the electrode structure can be formed from a plurality of resilient struts, wherein the active planar electrode surface 22 is secured to the front end of the struts. The struts may be collapsed inwardly by drawing shaft 24 rearwardly relative to the sheath 20, thus drawing the electrode structure 18 into the cannula. The electrically conductive surfaces 22, 23 will be connected to the power supply controller 14 through connecting cables 26 and 16. Usually, one connector will be provided for electrically isolated region 22 and another connector will be provided for electrically isolated region 23, as described in more detail below. It will be appreciated that the following depictions are for illustration purposes only and does not necessarily reflect the actual shape, size, or dimensions of the cardiac electrode system 10. This applies to all depictions hereinafter.

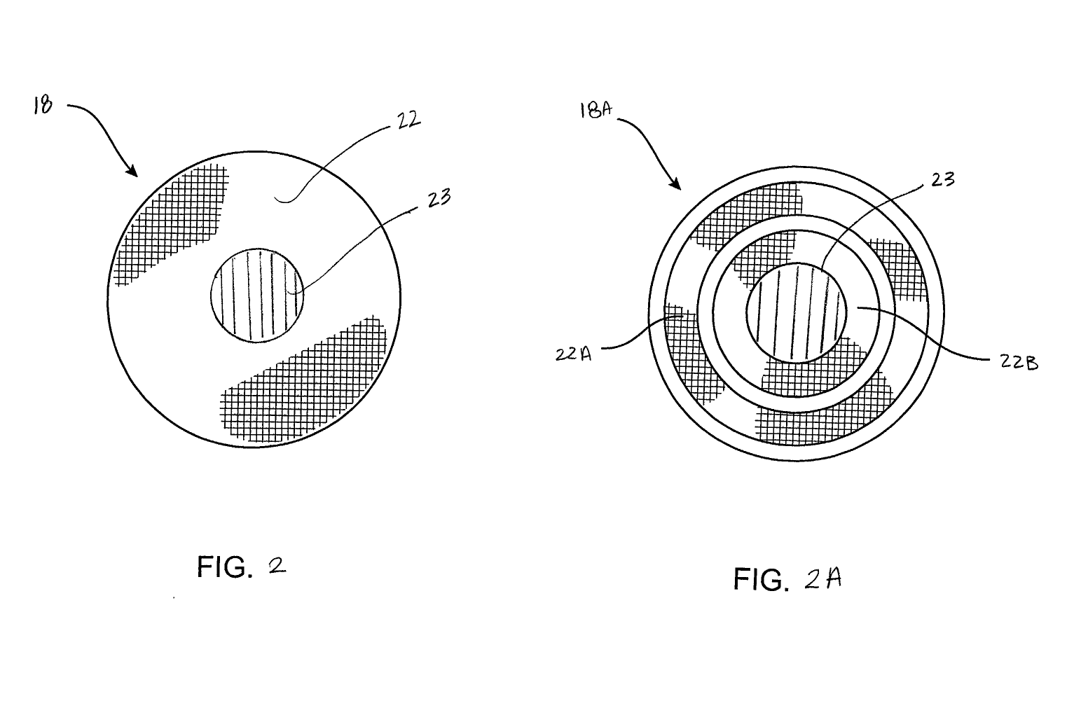

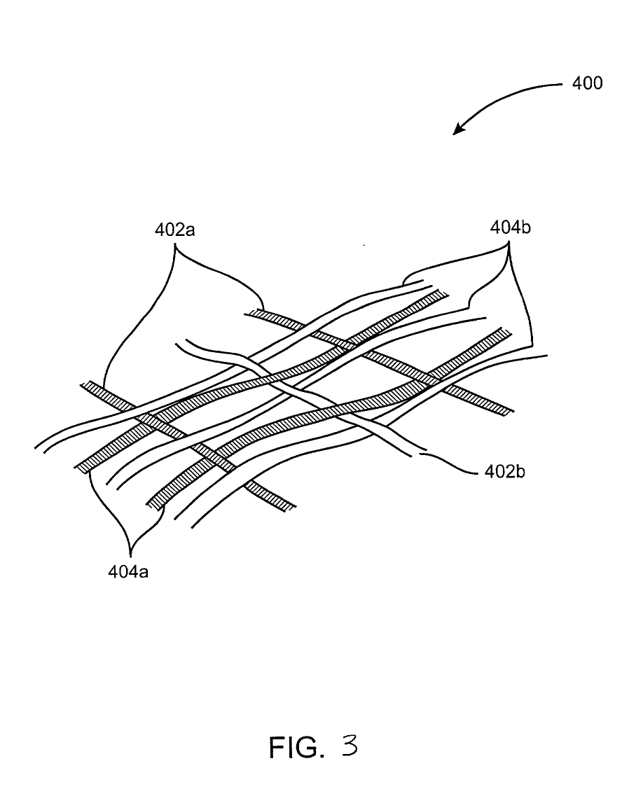

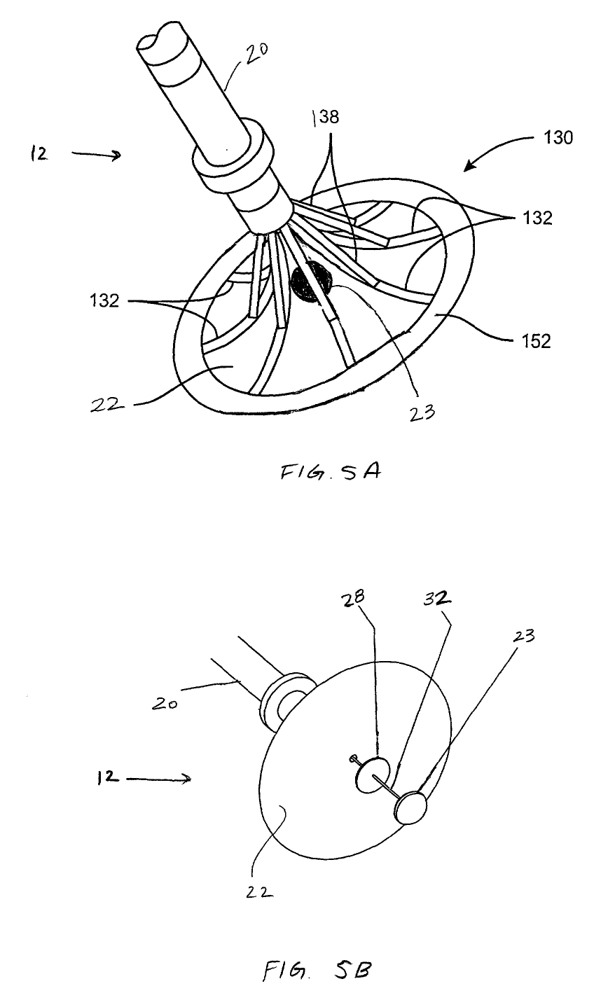

Referring now to FIG. 2, the electrode structure 18 comprises an electrically conductive base 22 having a generally circular periphery, although other peripheral geometries, such as ovoid, rectangular, triangular, irregular, and the like, could also be utilized. In FIG. 2, the electrically conductive base 22 comprises a compliant web which may be formed in any of the ways described above, and has an active electrode surface area in the range from 10 cm² to 300 cm², preferably from 32 cm² to 150 cm². The electrode structure 18 further comprises an electrically conductive dome 23 which may be a soft matrix or mesh attached to and projecting from the web 22 by an insulative spacer 28 (FIG. 4C). The dome 23 has an active electrode surface area in the range from 0.06 cm² to 20 cm², preferably from 10 cm² to 20 cm², where a smaller dome electrode surface may improve sensing and pacing capabilities. The convex surface of the domed electrode structure 23 is advantageous since its protruding surface can ensure enhanced electrical contact with the heart which in turn ensues reliable EKG and pacing signal acquisitions. The soft matrix of the conductive dome 23 further ensures increased surface contact with the heart, especially in circumstances where the electrode structure is deployed at odd angles against the heart. FIG. 2A illustrates an alternative electrode configuration 18A, where a pair of concentric ring electrode regions 22A and 22B are spaced-apart on the exposed planar surface 22 in addition to the raised central region 23. The three isolated electrode regions may be electrically isolated from each other and connected independently through shaft 24 by isolated electrical connectors.

It will be appreciated that the electrode regions 22 and 23 can be formed from a wide variety of conformable, electrically conductive materials or composites. Usually, the materials will be flexible but non-distensible, most usually being formed from non-distensible fabric or mesh. In one instance, the fabrics can be metalized, for example by vapor deposition or plating (either electro or electroless) of a conductive metal surface over a fabric or matrix. More usually, however, the conductive fabrics will be formed by weaving at least part of the fabric from a metal, preferably in both directions of the weave, but in some cases only in a single direction. The metal filaments in the fabric may be disposed at each strand or fiber, optionally at every other strand or fiber, usually will be placed at least once every 100 strands or fibers, more usually at least every tenth strand. The other strands or fibers may be formed from electrically non-conductive materials, such as polyester.

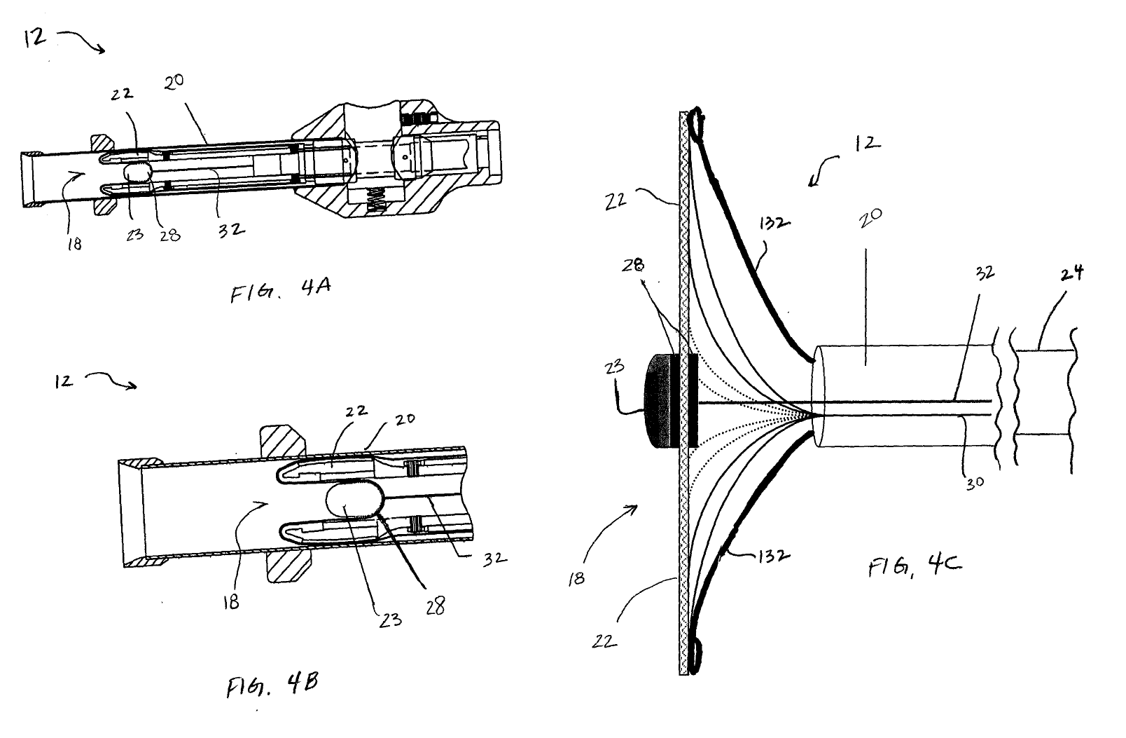

An exemplary fabric is illustrated in FIG. 3. The fabric 400 comprises warp 402 and woof 404 threads which are woven at right angles in a conventional pattern. Preferably, at least some of the warp threads 402 and the woof threads 404 will be electrically conductive, most preferably being a metal, such as titanium, gold, silver, stainless steel, copper, or other electrically conductive medically acceptable metal. In the exemplary structure, the conductive and non-conductive threads will be arranged in an alternating pattern as illustrated. Such an alternating construction provides very uniform strength and electrical conductivity characteristics.

Referring now to FIGS. 4A-4C, an insulative spacer 28 made from rubber, plastic, or like insulative materials, attaches the dome 23 to the compliant web 22 and ensures that the planar 22 and center regions 23 are sufficiently separated and that the dome 23 remains centered. Preferably, the electrode structure 18 on the deployment device 12 will be capable of being shifted between a low profile configuration suitable for intercostal introduction to a region over the patient's heart, as shown in FIGS. 4A and 4B, and to an open configuration where the electrode surfaces 22 and 23 can be engaged against the heart, as shown in FIG. 4C. The electrode surfaces 22 and 23 of the electrode structure comprise two electrically isolated segments which can be separately but simultaneously energized from opposite poles of the external power supply controller 14 (see FIG. 1), allowing bipolar functioning as described in more detail below. The support 24 will usually include separate electrical conduction paths for connecting the isolated segments of the electrode structure to the external power supply controller 14. FIG. 4C further illustrates the electrical connections of the device 12, wherein the support 24 comprises a first electrically conductive path 30 for connecting the electrode surface on the planar region 22 to the external power supply 14 through connecting cable 26 (see FIG. 1) and a second electrically conductive path 32 isolated from the first path 30 for connecting the electrode surface of the center region 23 to the external power supply controller 14 through connecting cable 16 (see FIG. 1).

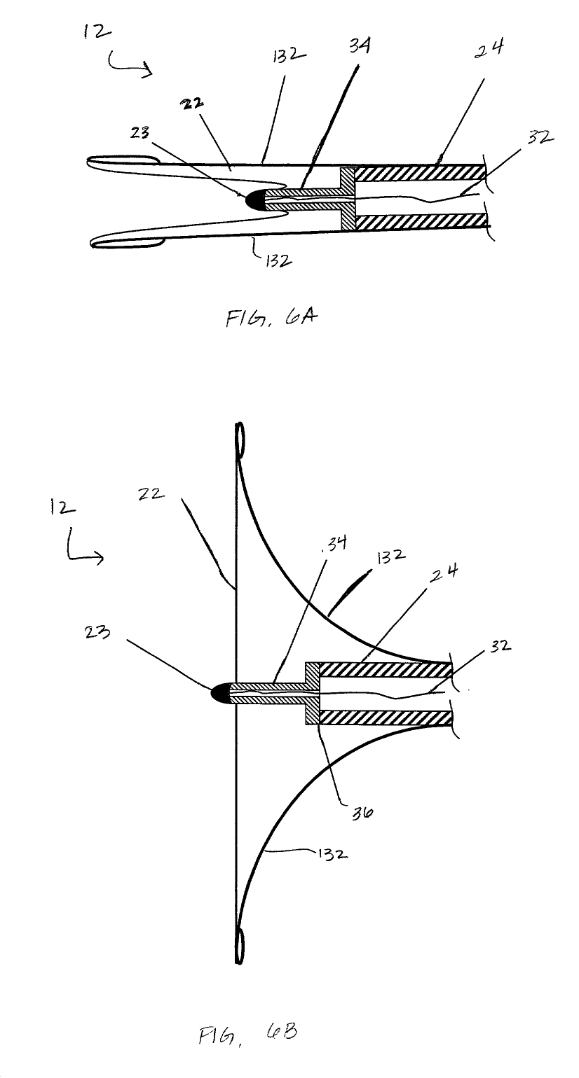

Referring now to FIGS. 5A and 5B, in an exemplary embodiment, the compliant web 22 is mounted to a flared bell structure 130 that is attached to the distal end of the shaft 24 and assumes a trumpeted configuration when fully deployed. The flared bell structure 130 comprises a plurality of outwardly curving struts 132 (the illustrated embodiment has a total of eight struts, but this number could vary). The struts are retractable to a radially contracted configuration and advanceable along arcuate, diverging paths to define a surface which non-traumatically engages the pericardium to compress the heart when advanced against the pericardium. The struts are preferably formed from a resilient metal, usually formed from a superelastic alloy, such as nitinol. To enhance the rigidity and pushability of the structure, re-enforcing beams 138 may be provided. It has been found that the combination of the curved struts with straight beam supports provides a useful combination of stiffness over the proximal portion of the structure and greater flexibility at the tip portions.

The compliant web or conductive fabric 22 will be secured to the struts 132 to advance the electrode surfaces 22 and 23 when the struts are advanced. The distal tips of the struts 132 are preferably connected by the fabric electrode structure 22 having an edge which is folded over and stitched to hold the fabric in place. In this embodiment, the compliant web 22 is supported solely by the struts 132 and the soft, conductive dome 23 is supported solely by the web 22.

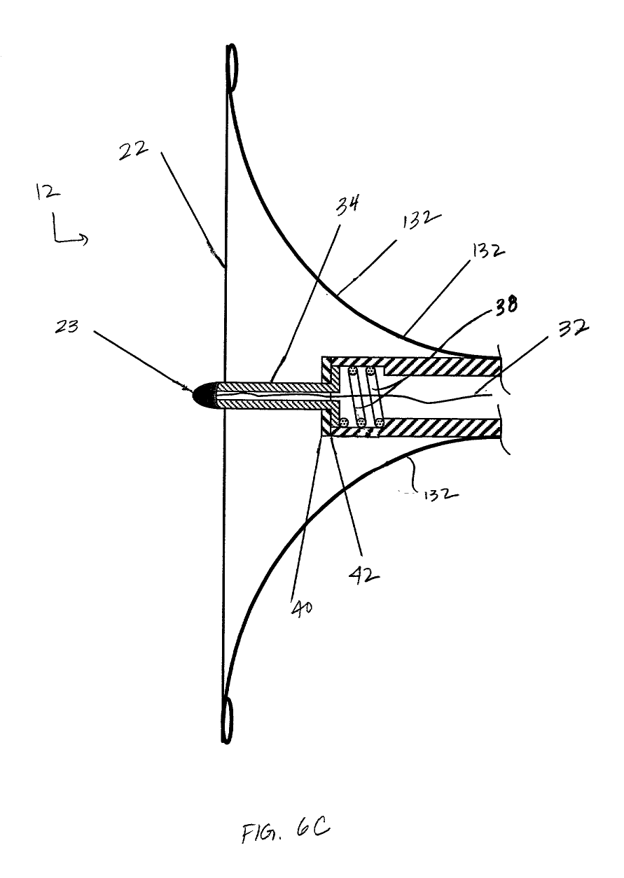

Referring now to FIGS. 6A and 6B, in some instances the deployment device 12 may further incorporate a non-conductive, fixed rod 34 which is advancable from a distal end of the shaft 24 to urge the dome 23 to protrude distally of the compliant web 22 as the electrode surfaces are deployed (FIG. 6B) against the heart to maximize electrode surface contact with the heart. In such an embodiment, one end of the fixed rod 34 is attached to the distal end of the shaft 24 by bonded or threaded mating surfaces 36 and the other end is attached to the dome 23 and the compliant web 22. The fixed rod 34, made from rigid plastic, rubber, or like insulative materials, further ensures that the planar and center regions are sufficiently separated and that the dome remains centered. Referring now to FIG. 6C, the deployment device 12 may additionally include a spring mechanism 38 attached to the distal end of the shaft 24 and engageable with the fixed rod 34 to provide a spring loaded advancement of the fixed rod 34 which in turn urges the dome 23 to protrude distally of the compliant web 22. The fixed rod 34 in this case may be secured within the distal end of the shaft 24 by a shaft cap 40 and bonded or threaded mating surfaces 42.

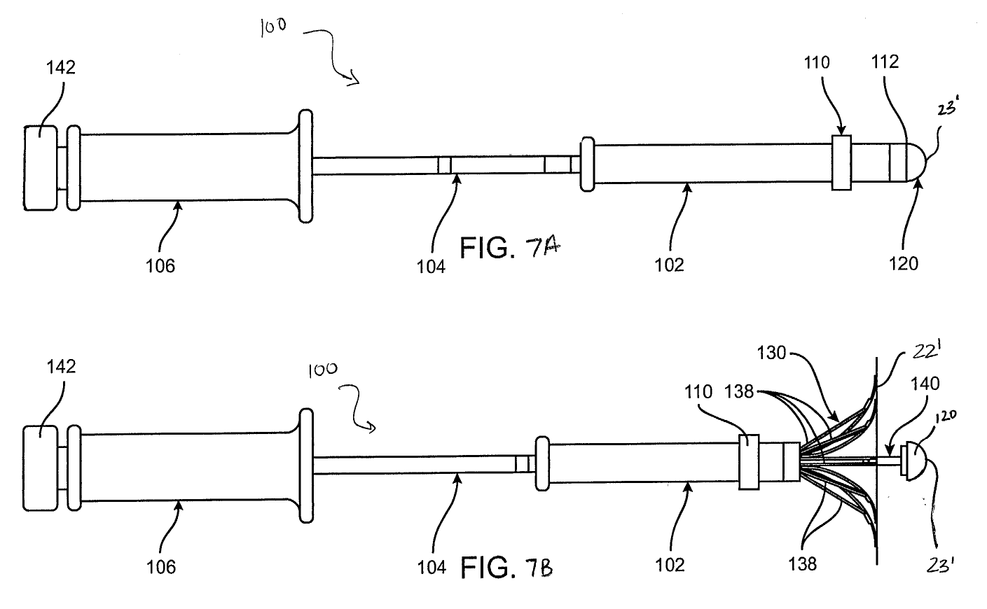

Referring now to FIGS. 7A and 7B, an alternative cardiac electrode deployment device 100 comprises a sleeve 102, a shaft 104 slidably mounted in a central lumen of the sleeve 102, and a handle 106 attached to a proximal end of the shaft. The sleeve 102 includes a positioning flange 110 near its distal end, typically spaced proximally of the tip 112 of the device. A blunt tip 120 is positioned at the distal-most end of the device 100 and facilitates entry of the device into the chest cavity by blunt dissection, as described in more detail hereinafter. A first electrode structure 22′ may be deployable from the distal end of the shaft 104, wherein the first electrode structure 22′ includes a planar region. A second electrode structure 23′ may be attached to the blunt tip 120, wherein the second electrode structure has a conformable, raised center region, and electrode surfaces on the first and second electrode structures 22′ and 23′ are electrically isolated from each other. In this embodiment, the first electrode structure 22′ comprises an electrically conducive base, such as a compliant web or fabric as mentioned above. The second electrode structure 23′ comprises an electrically conductive dome, such as a soft matrix or mesh, disposed over the blunt tip 120. The device 100 differs principally in that it includes a blunt tip 120 that advantageously allows for both intercostal dissection and sensing or pacing of the heart.

The compliant web 22′ may be mounted on a flared bell structure 130 as described above. The compliant web 22′ is secured to the struts 132 to advance the first electrode surface 22′ when the struts are advanced. The compliant web 22′ in this embodiment is supported solely by the struts and the dome 23′ is supported solely by the blunt tip 120. The blunt tip 120 is mounted on a rod 140 having an electrical connector 142 at its proximal end. The isolated electrode surfaces are electrically connected through a plurality of conductors (not shown) which terminate in the electrical connector 142. The connector 142 will typically include an array of plug prongs or receptacles which permit inner connection of the connector with a cable which in turn connects the device to a suitable power supply controller. When the sleeve 102 is advanced distally over the flared bell structure 130, the forward tip of the sleeve will engage the rear of the blunt tip 120, as seen in FIG. 7A. When the sleeve 102 is retracted and the flared bell structure 150 deployed, the blunt tip 120 will be free to move axially, as best seen in FIG. 7B. The blunt tip 120 may be formed from a soft, biocompatible foam core or may optionally be entirely formed from soft conductive mesh. The deployment device 100 of the present invention may further incorporate a force gauge, accelerometer, impedance sensor, piezoelectric crystal, or oximeter into the blunt tip or dome to further allow sensing of other characteristics (not shown).

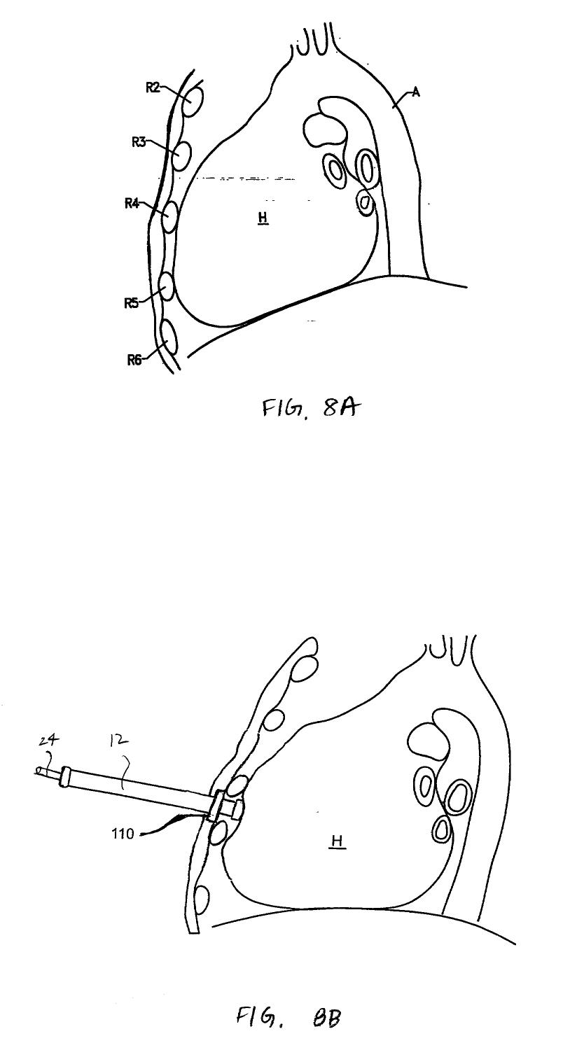

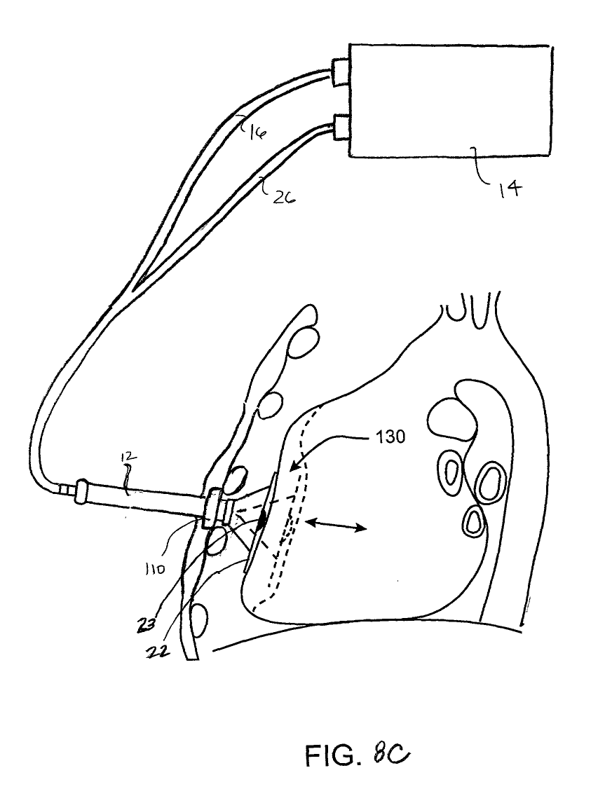

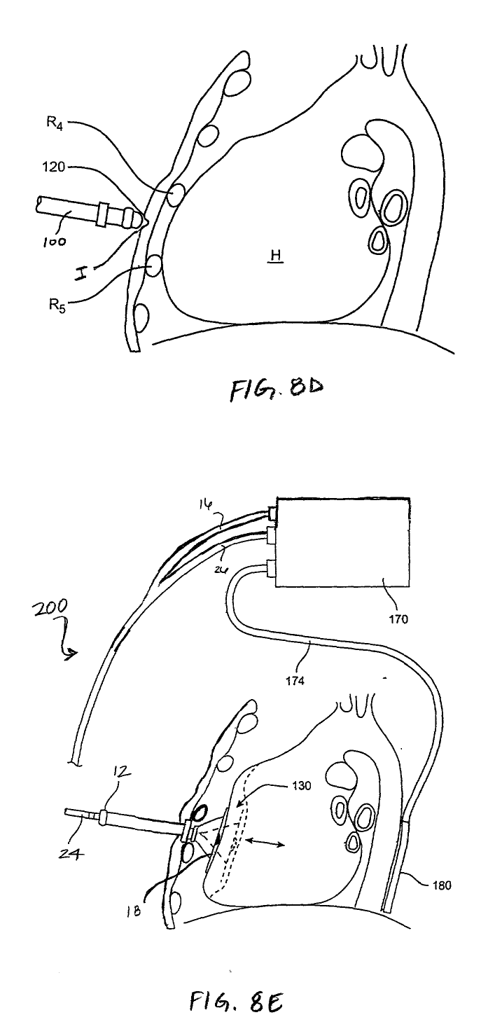

Referring now to FIGS. 8A-8C, an electrode deployment device can be introduced into a region over the heart and used for direct cardiac massage. A patient's heart H is shown in FIG. 8A in cross-section between ribs Rₙ where n indicates the rib number. The aorta A is also shown extending from the top of the heart. Initially, intercostal access into the chest cavity may be established by sharp dissection or a combination of sharp and blunt dissection, preferably on the patient's left side between the fourth and fifth ribs (R₄ and R₅). After an intercostal access tract is established, the device 12 is pushed through the access tract until the flange 110 engages the ribs, as illustrated in FIG. 8B. At that point, the flared bell structure is still not deployed. The flared bell structure 130 is then deployed by advancing the shaft 24. Once the structure 130 is fully deployed, the shaft may be manually pumped through the sheath. This will cause the deployed flared bell structure 130 to engage the planar 22 and domed 23 electrode surfaces against the heart. The structure can then be advanced in a posterior direction to compress the heart, generally shown in broken line in FIG. 8C. Preferably, the shaft will be inclined from 20° to 45° toward the patient's left in the mid-sagittal plane while being held generally vertically in the cranial-caudal plane. In this way, the electrode surfaces compress the heart toward the patient's spine to maximize compression.

A first electrically conductive path is established by engaging the electrically compliant web 22 against the heart and a the second electrically conductive path is established by engaging the soft dome-like matrix 23 attached to and projecting from the web against the heart (FIG. 5A). Alternatively, the first electrically conductive path may comprise engaging an electrically conductive compliant web 22 against the heart and establishing the second electrically conductive path may comprise engaging a soft dome-like matrix 23 disposed over a blunt tip against the heart (FIG. 7B). In the latter case, the blunt tip 120 may facilitate introduction of the electrode structure by bluntly dissecting intercostal tissue after an initial small incision I is made over the heart, as shown in FIG. 8D. An electrical circuit is then established using the external power supply 14 which is connected via cable 26 to the planar electrode structure 22 on the flared bell structure 130 and a cable 16 to the dome electrode structure 23 on the web. Energy is applied according to the protocols described below. Once resuscitation has been completed, the device 120 may be withdrawn by retracting the shaft 24 relative to sheath 20 to draw the structure 130 back into the sleeve. Once the structure 130 is retracted, the device may be proximally withdrawn through the incision and the incision closed in the conventional manner.

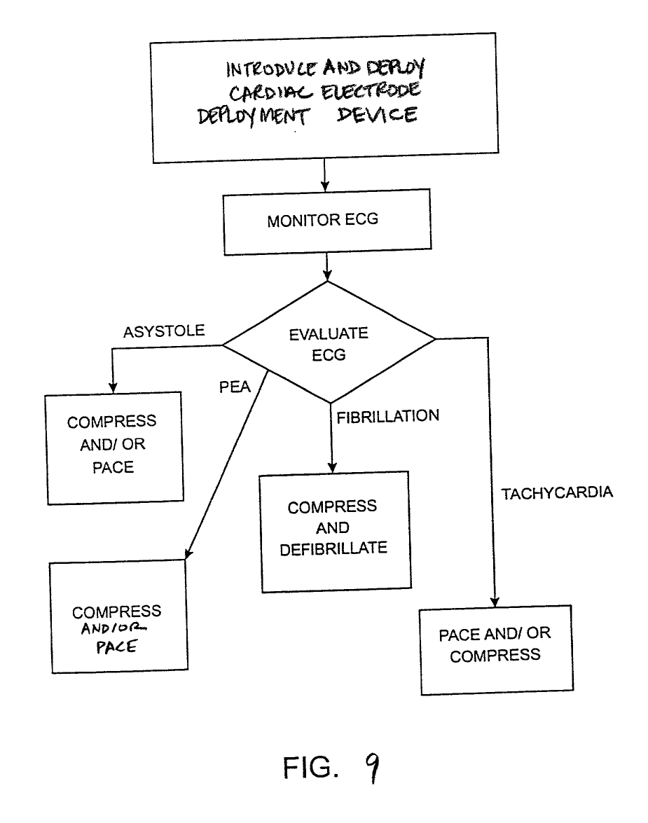

A preferred protocol for utilizing the cardiac electrode deployment device to resuscitate a patient in cardiac arrest is shown in FIG. 9. After the cardiac electrode device is introduced and deployed, as generally shown in FIGS. 8A-8C, the patient's EKG/ECG will be monitored. Preferably, the planar 22 and central 23 isolated electrode surfaces in contact with the heart will be used for monitoring. Based on the nature of the patient's EKG/ECG, a preferred course of treatment can be selected. If the patient is determined to be in asystole, i.e., no cardiac sinus rhythm, then the patient will be treated by compression optionally with pacing. Compression and/or pacing may be carried out by applying bipolar energy through the planar 22 and central 23 isolated electrode surfaces and can be continued until a normal cardiac rhythm is reestablished or it is determined that the patient cannot be resuscitated. If the patient is determined to be in tachycardia, then the device will be used to apply pacing energy directly to the heart. Optionally, the heart can be compressed simultaneously and more preferably synchronously, with the application of pacing energy. If the patient is determined to be in pulseless electromechanical activity, a preferred course of treatment will be compression optionally with pacing. Finally, if the patient is determined to be in fibrillation, defibrillation energy will be applied to the heart as described below (FIG. 8E). Compression can be performed simultaneously and/or after the heart has been defibrillated. It should be further noted that prior to, during, or immediately following such compression, the electrode surfaces on the device may be used to monitor the patient ECG.

Use of a modified device 200 for applying defibrillation energy is illustrated in FIG. 8E. The device differs principally in that it includes a counter or dispersive electrode 180 and an external power supply 170 that further contains circuitry necessary for producing the defibrillation energy. Usually, defibrillation energy will be applied to the patient in a unipolar fashion, wherein typically the patient's back is engaged with the counter electrode 180 and defibrillation energy is applied between the electrode structure 18 on the heart (planar electrode region 22 via cable 26 connected to power supply 170 or center electrode region 23 via cable 16 connected to power supply 170) and the counter electrode 180 on the back (via cable 174 connected to power supply 170) so that direct current countershock can be applied to the heart along an anterior-posterior direction. Typically, defibrillation will require a total amount of energy in the range from 2 joules to about 100 joules, usually from 20 joules to 50 joules for biphasic defibrillation. Hence, the present invention may advantageously pace or sense through the planar or center regions and defibrillate through the planar/center region in combination with the counter electrode.

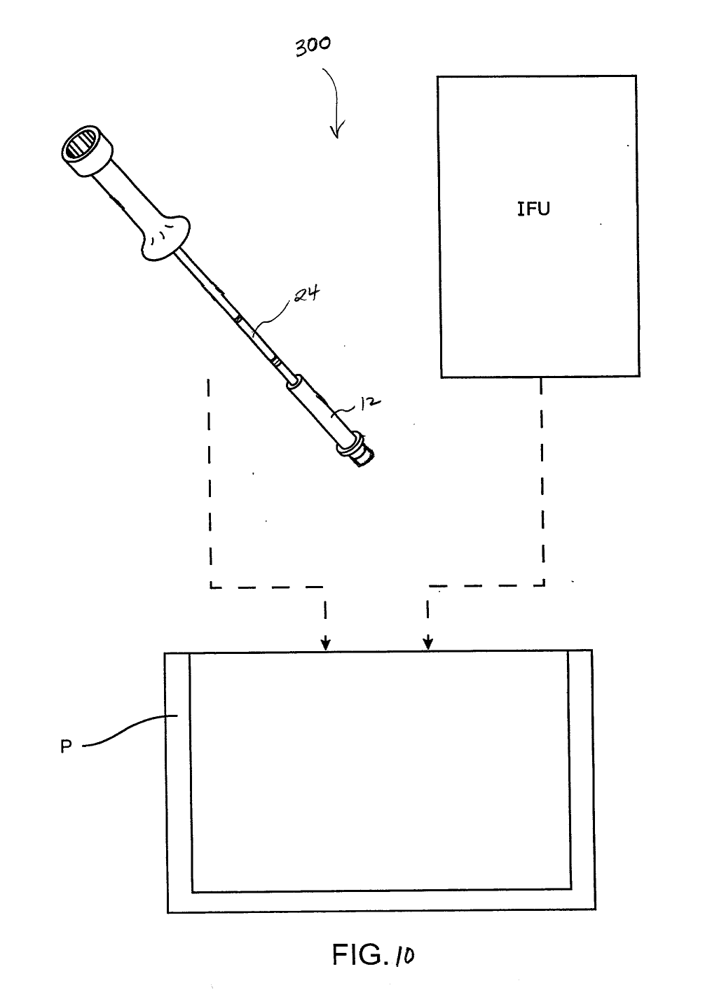

Referring now to FIG. 10, a kit 300 according to the present invention comprises a cardiac electrode deployment tool, such as device 12 described in detail previously, in combination with instructions for use IFU setting forth any of the methods described above. Usually, the device and instructions for use will be combined in a suitable package P that can be in the form of any conventional medical device packaging, such as a tray, tube, box, pouch, or the like. The instructions for use will usually be provided on a separate package insert, but could also be printed directly on all or a portion of the packaging P. Additional components, such as a counter electrode, could also be provided as part of the kit.

Although certain preferred embodiments and methods have been disclosed herein, it will be apparent from the foregoing disclosure to those skilled in the art that variations and modification of such embodiments and methods may be made without departing from the true spirit and scope of the invention. Therefore, the above description should not be taken as limiting the scope of the invention which is defined by the appended claims.

What is claimed is:

1. A cardiac electrode deployment device comprising:
a support; and
an electrode structure deployable from the support, said electrode structure including a planar region and a conformable, raised center region, wherein electrode surfaces on the planar region and on the center region are electrically isolated from each other.

2. A device as in claim 1, wherein the electrode structure comprises an electrically conductive base and an electrically conductive dome attached to an electrically insulative spacer from the base.

3. A device as in claim 2, wherein the electrically conductive base is a compliant web and the conductive dome is a soft matrix attached to and projecting from the web.

4. A device as in claim 3, wherein the electrode structure can be shifted between a low profile configuration where it can be intercostally introduced to a region over the heart and an open configuration where the electrode surfaces can be engaged against the heart.

5. A device as in claim 4, wherein support comprises a shaft having a proximal end and a distal end and the electrode structure comprises a plurality of struts reciprocatably attached to the distal end of the shaft, said struts being retractable to a radially contracted configuration and advancable along arcuate, diverging paths to deploy the electrode surfaces to non-traumatically engage the heart when advanced thereagainst, wherein the compliant web is secured to the struts to advance the electrode surfaces when the struts are advanced.

6. A device as in claim 5, wherein the compliant web is supported solely by the struts and the dome is supported solely by the web.

7. A device as in claim 1, further comprising a non-conductive, fixed rod which is coupleable to the center region and advancable from a distal end of the support to urge the center region forward as the electrode surfaces are advanced against the heart.

8. A device as in claim 7, further comprising a spring attached to the distal end of the support to provide a spring loaded advancement of the fixed rod.

9. A device as in claim 1, wherein at least one of the electrode surfaces of the electrode structure comprises a plurality of electrically isolated segments and wherein the support includes separate electrical conduction paths for connecting the isolated segments of the electrode structure to an external power supply controller.

10. A device as in claim 1, wherein the support comprises a first electrically conductive path for connecting the electrode surface on the planar region to an external power supply controller and a second electrically conductive path isolated from the first path for connecting the electrode surface of the center region to the external power supply controller.

11. A system comprising:
a support;
an electrode structure deployable from the support, said electrode structure including a planar region and a conformable, raised center region, wherein electrode surfaces on the planar region and on the center region are electrically isolated from each other;
a power supply controller; and
wherein the support comprises a first electrically conductive path for connecting the electrode surface on the planar region to the external power supply controller and a second electrically conductive path isolated from the first path for connecting the electrode surface of the center region to the external power supply controller.

12. A system as in claim 11, further comprising a paired counter electrode.

13. A system as in claim 12, further comprising a switch on the power supply controller to allow a user to switch the mode of operation between bipolar functioning for sensing or pacing treatment and unipolar functioning for defibrillation treatment.

14. A cardiac electrode deployment device comprising:
a support having a proximal end, a distal end, and a blunt tip;
a first electrode structure deployable from the distal end of the support, said first electrode structure including a planar region; and
a second electrode structure attached to the blunt tip, said second electrode structure having a conformable, raised center region, wherein electrode surfaces on the first and second electrode structures are electrically isolated from each other.

15. A device as in claim 14, wherein the first electrode structure comprises an electrically conductive base and the second electrode structure comprises an electrically conductive dome.

16. A device as in claim 15, wherein the electrically conductive base is a compliant web and the conductive dome is a soft matrix or mesh disposed over the blunt tip.

17. A device as in claim 16, wherein the first electrode structure comprises a plurality of struts reciprocatably attached to the distal end of the shaft, said struts being retractable to a radially contracted configuration and advancable along arcuate, diverging paths to deploy the first electrode surface to non-traumatically engage the heart when advanced thereagainst, wherein the compliant web is secured to the struts to advance the first electrode surface when the struts are advanced.

18. A device as in claim 17, wherein the compliant web is supported solely by the struts and the dome is supported solely by the blunt tip.

19. A device as in claim 18, wherein the blunt tip extends from the most distal end of the shaft by a rod.

20. A device as in claim 19, wherein the blunt tip is formed from a soft, biocompatible foam.

21. A device as in claim 19, wherein the blunt tip is formed entirely from a soft conductive mesh.

22. A device as in claim 19, further comprising a force gauge, accelerometer, impedance sensor, piezoelectric crystal, or oximeter coupled to the blunt tip or dome.

23. A method for electrically contacting a heart, said method comprising:
percutaneously introducing an electrode structure against the heart;
establishing a first electrically conductive path to the heart through a first electrode surface on a planar region of the electrode structure;
establishing a second electrically conductive path to the heart through a second electrode surface on a raised center region of the electrode structure, wherein the first and second electrode surfaces are electrically isolated from each other; and
establishing an electrical circuit between the first and second electrically conductive paths.

24. A method as in claim 23, wherein establishing a circuit comprises taking an EKG of the heart.

25. A method as in claim 23, wherein establishing a circuit comprises pacing the heart.

26. A method as in claim 23, wherein establishing a circuit comprises applying energy in a bipolar fashion through the first and second isolated electrode surfaces.

27. A method as in claim 23, wherein establishing the first electrically conductive path comprises engaging an electrically conductive compliant web against the heart and establishing the second electrically conductive path comprises engaging a soft dome-like matrix coupled to and projecting from the web against the heart.

28. A method as in claim 27, further comprising advancing the dome-like matrix to protrude distally of the compliant web.

29. A method as in claim 23, wherein the first electrically conductive path comprises engaging an electrically conductive compliant web against the heart and establishing the second electrically conductive path comprises engaging a soft dome-like matrix disposed over a blunt tip against the heart.

30. A method as in claim 29, wherein introducing the electrode structure comprises bluntly dissecting intercostal tissue with the blunt tip.

31. A method as in claim 23, further comprising compressing the heart by contacting the electrode structure against the heart and pressing the electrode structure to cause compression of the heart.

32. A method as in claim 31, wherein compression is in an anterior-posterior direction.

33. A method as in claim 31, wherein the electrode structure is introduced intercostally in a low profile configuration and subsequently expanded over the heart.

34. A method as in claim 31, wherein compressing the heart comprises repetitively compressing the heart at from 40 to 160 repetitions per minute.

35. A method as in claim 23, further comprising contacting a patient's back with a counter electrode and applying defibrillation energy between the electrode structure on the heart and the counter electrode on the patient's back to defibrillate the heart.

36. A method as in claim 35, wherein applying defibrillation energy comprises switching the mode of operation on a power supply connected to the electrode structure and the counter electrode.

37. A kit comprising;
a cardiac electrode deployment device; and
instructions for use setting forth a method according to claim 23.
